(19) United States
(12) Patent Application Publication
(10) Pub. No.: US 2012/0070858 A1
(43) Pub. Date: Mar. 22, 2012

(54) METHOD FOR ISOLATING EXOSOMES FROM BIOLOGICAL SOLUTIONS USING IRON OXIDE NANOPARTICLES

(76) Inventors: Lionel Fernel Gamarra Contreras, Sao Paulo (BR); Daiane Dona Guilhen, Sao Paulo (BR); Mariano Janiszewski, Sao Paulo (BR); Lonena Favaro Pavon, Sao Paulo (BR); Luciana Cavalheiro Marti, Sao Paulo (BR)

(21) Appl. No.: 13/266,076

(22) PCT Filed: Jan. 28, 2010
(86) PCT No.: PCT/BR2010/000032

§ 371 Date: Nov. 21, 2011

(30) Foreign Application Priority Data
Apr. 23, 2009 (BR) PI0900815-2

(51) Int. Cl. C12Q 1/02; C12N 13/00

(52) U.S. Cl. 435/29; 435/173.1

(57) ABSTRACT

A method for isolating exosomes from blood platelets using superparamagnetic nanoparticles of iron oxide (Fe₃O₄), by means of a charge attraction mechanism based on the predetermined Zeta potential of the exosomes. The method involves the use of iron oxide nanoparticles that are previously synthesised with a predetermined positive charge, and that bond to the negatively charged exosomes contained in the biological sample. During incubation, the cationic magnetic nanoparticles are absorbed by the surface of the membrane of the exosomes owing to electrostatic interaction. Exposure of the material to a magnetic field makes it possible to separate the exosomes bonded to the nanoparticles. The success of this technique has been confirmed by characterisation of the exosomes by flow citometry. The method has been shown to be suitable for this purpose, since it allows exosomes to be isolated and purified, without undergoing alterations of their original morphological and structural characteristics.

### APPLICATION FIELD

The present invention relates to a method for isolation of exosomes derived from platelets using superparamagnetic magnetite nanoparticles (Fe₃O₄), through an attraction charge mechanism based on the exosomes pre-determined zeta potential.

### BACKGROUND OF THE INVENTION

Exosome is a type of microparticle produced by different types of regular and tumor cells (lymphocytes, platelets, dendritic cells, neuron cells, mast cells, intestinal cell, macrophages, among others) with an ample signal function. It has 100 nm of diameter approximately, it is composed by a double lipid layer associated with a membrane of proteins, containing proteins internally, nucleic acid and lipids. Recent studies show an ample capacity of inducing a more efficient immune response, as well as promoting immune tolerance. Such properties made that the preparation of anti-tumor vaccines based on cell exosomes previously sensitized were proposed. More recently, exosomes showed that they were able to promote angiogenesis, apoptosis of vascular cells, dysfunction of cardiac cells and to even transmit genetic information among cells. Also, it is believed that they may transmit prion and mycobacterial disease.

Despite the evident importance of the exosomes, their split and isolation from biological substances, preserving their structural and functional integrity to study and use, represents a problem. The conventional isolation of exosomes is very difficult, slow and does not guarantee structural conservation of the particles. From the original biological solution, a series of centrifugations are done, which guarantee precipitation of cells, debris and particles due to its relative density and size.

Within a standard protocol, in order to obtain exosomes from a cell solution, the solution is submitted to centrifugation of: 1000 g during 15 minutes to remove cells and huge debris; then continue at 4° C. at 18,000 g during 30 minutes to remove bigger subcell particles, apoptotic bodies and undesirable organelles; immediately, the supernatant of microvesicular fraction is sequentially filtered through nylon membranes of 1 μm, 500 nm and 220 nm, and then, centrifugated again at 4° C. at 100,000 g during 90 minutes in order to obtain the exosomes “pellet”. Such pellet may be suspended again for its use.

All results herein have been obtained from exosomes originated from platelets. Previous studies show that in a clinic situation of sepsis, platelets exosomes may be related to vascular and cardiac dysfunction. These exosomes express CD63 (tetraspanin) in abundance, and show weakly annexin V on their surface.

For a better understanding of the principle of the method for isolation of exosomes derived from nanoparticles of iron oxide used in this invention, a brief explanation about “zeta potential” is necessary.

Almost all macroscopic or particulate materials in contact with a liquid acquire an electric charge on its surface. Such charge may appear from the dissociation of ions on the particle surface, the differential adsorption of ions of the solution on the particle surface, among others. The liquid charge on the particle surface affects the distribution of ions in its neighborhood, increasing the concentration of counter-ion on the surface. Thus, an electric double layer is formed on the particle interface with the liquid.

Such double layer is split in two regions: an internal region including ions strongly linked to the surface and an external region where the distribution of ions is determined by the balance between electrostatic forces and thermic movement. In this way, the potential in this region decreases with the increase of distance from the surface, in an enough long distance, to reach the solution potential. Such potential is stipulated as zero potential.

In an electric field, each particle and the most strongly ions linked to it move themselves as a unit, and the potential in the interface plan between such unit and the environment is called zeta potential. Therefore, zeta potential is a useful indicator of such charge which may be used to foresee and control the suspensions stability or colloid emulsions. The bigger the zeta potential the more probable the suspension may be stable due to the fact the charged particles are repulsed ones to the others and such force outdo the natural tendency to the aggregation.

Zeta potential cannot be measured directly. Thus, a type of indirect measure is used, from which zeta potential is calculated. The technique mostly used and accepted is through electrophoretic mobility, a colloid suspension diluted in a tub with two electrodes is instated and an electric potential to the suspension is applied. The particles with liquid electric charge will move into the direction of the electrode of opposed charge. The quotient of the displacement speed along the electric field is called electrophoretic mobility, expressed in m²/V.s. This value enters in an equation (the more used are Smoluchowski's or Debye's approximations) to calculate the Zeta Potential.

### BRIEF DESCRIPTION OF THE INVENTION

Analyzing the current state of the technique, the solicitant developed a method for isolation of exosomes derived from platelets using superparamagnetic magnetite nanoparticles (Fe₃O₄), through an attraction charge mechanism based on the exosomes pre-determined zeta potential.

The method, basically, consists of the use of iron oxide nanoparticles previously synthetized with pre-determined positive charge that are linked to the exosomes negatively charged, comprised in the biological sample, by means of electric attraction; the material exposure to a magnetic field allows the split of exosomes that were linked to the nanoparticles; the success of this technique is confirmed by the characterization of exosomes by flow cytometry.

The method showed to be adequate for such objective, once it allows exosomes to be isolated and purified, and alterations within the morphologic and structural original characteristics of exosomes were not observed.

### DESCRIPTION OF THE FIGURES

In order to complement the description aiming at obtaining a better understanding of the invention details, a detailed description of the current method is made and it is accompanied by figures which show the analysis that demonstrate the success of the new method.

FIG. 1—Flowchart that shows the new method for isolation of exosomes derived from biological substances using iron oxide nanoparticles.

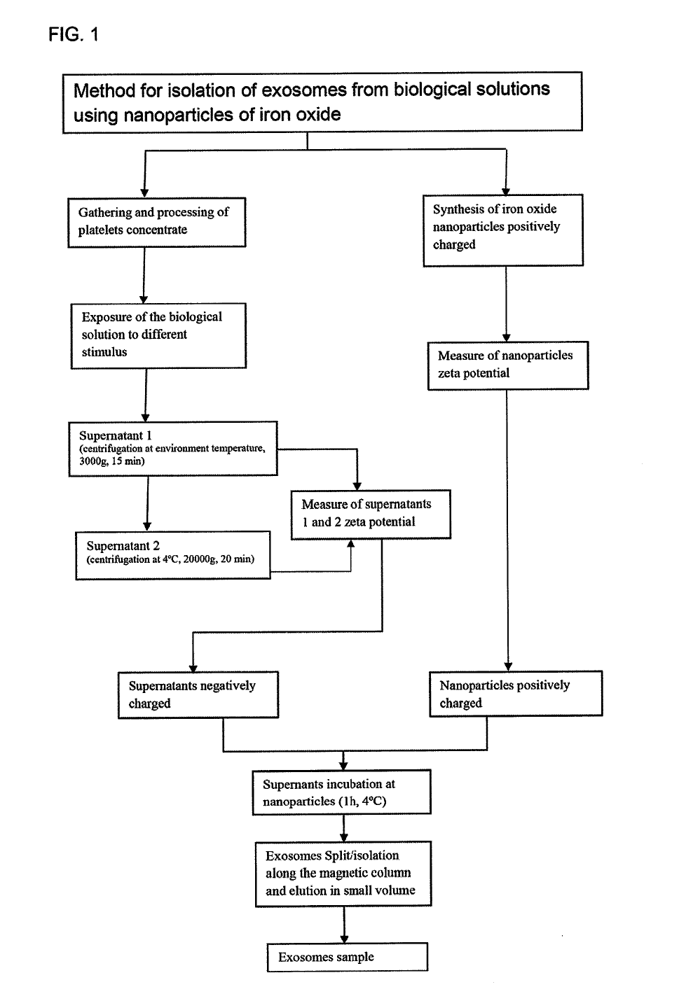

FIG. 2—Scheme of zeta potential principle functioning.

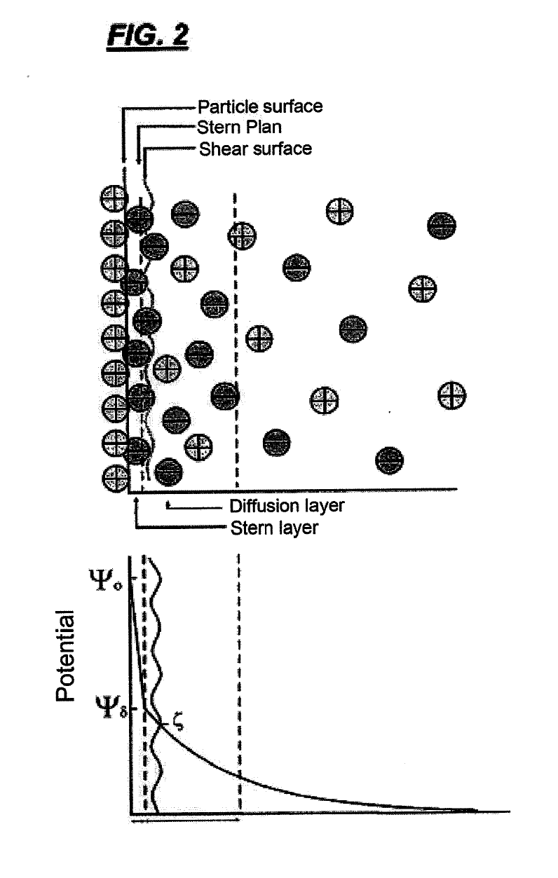

FIG. 3—Graph showing the differences between zeta potentials obtained from the measures made in microparticles derived from platelet degradation (PBS) and the exposure to thrombin (5 Ul/ml)—supernatant 1—versus exosomes obtained from the exposure of platelet to LPS (100 ng/ml)—supernatant 2—. Result of 4 independent experiments.

FIG. 4—Dot-plots showing the obtainment of corresponding fluorescent sign, in the first situation, merely detection of ferrous nanoparticles in PBS buffer with antibodies, showing the lack of meaningful fluorescence (artifact); and, in the second situation, the detection of the sample itself, having already taken out the “background” found in the first situation.

FIG. 5—Graphs clearly showing a differential detection by flow cytometry of the expression of the surface marker of exosomes, CD63, CD3, and CD9 and low expression of annexin V and HLA-DR, when compared with microparticles obtained by platelets degradation. In both situations, particles were obtained by ferrous nanoparticles.

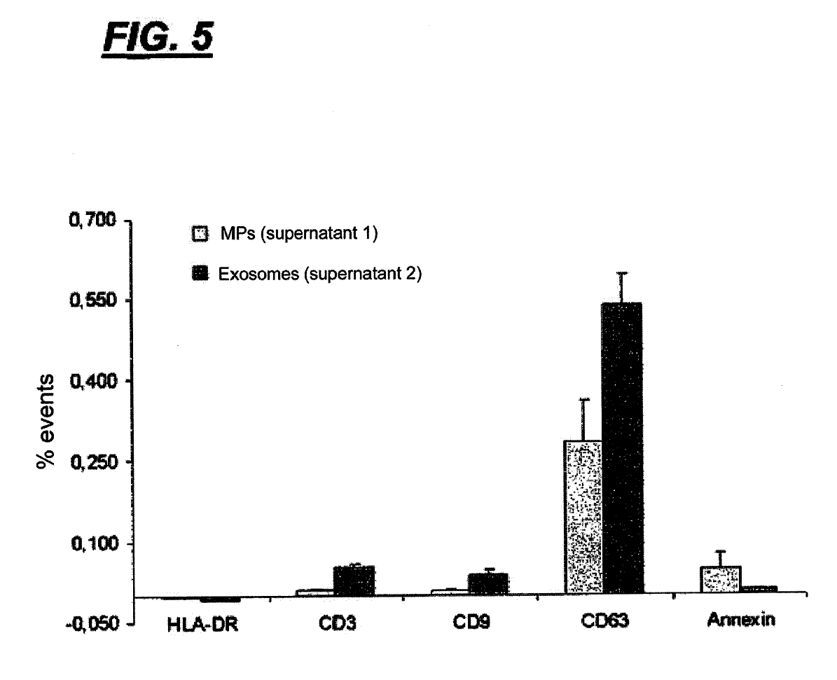

### DETAILED DESCRIPTION OF THE INVENTION

In reference to the figures, the current invention relates to a “METHOD FOR ISOLATION OF EXOSOMES FROM BIOLOGICAL SOLUTIONS USING NANOPARTICLES OF IRON OXIDE”, being that, more specifically, the method for isolation of exosomes derived from platelets using superparamagnetic magnetite nanoparticles (Fe₃O₄), is done through an attraction charge mechanism based on the exosomes pre-determined zeta potential.

The method consists of the use of iron oxide nanoparticles previously synthetized with pre-determined positive charge that are linked to the exosomes negatively charged, comprised in the biological sample, by means of electric attraction; the material exposure to a magnetic field allows the split of exosomes that were linked to the nanoparticles; the success of this technique is confirmed by the characterization of exosomes by flow cytometry.

Therefore, the method can be defined in the following stages:

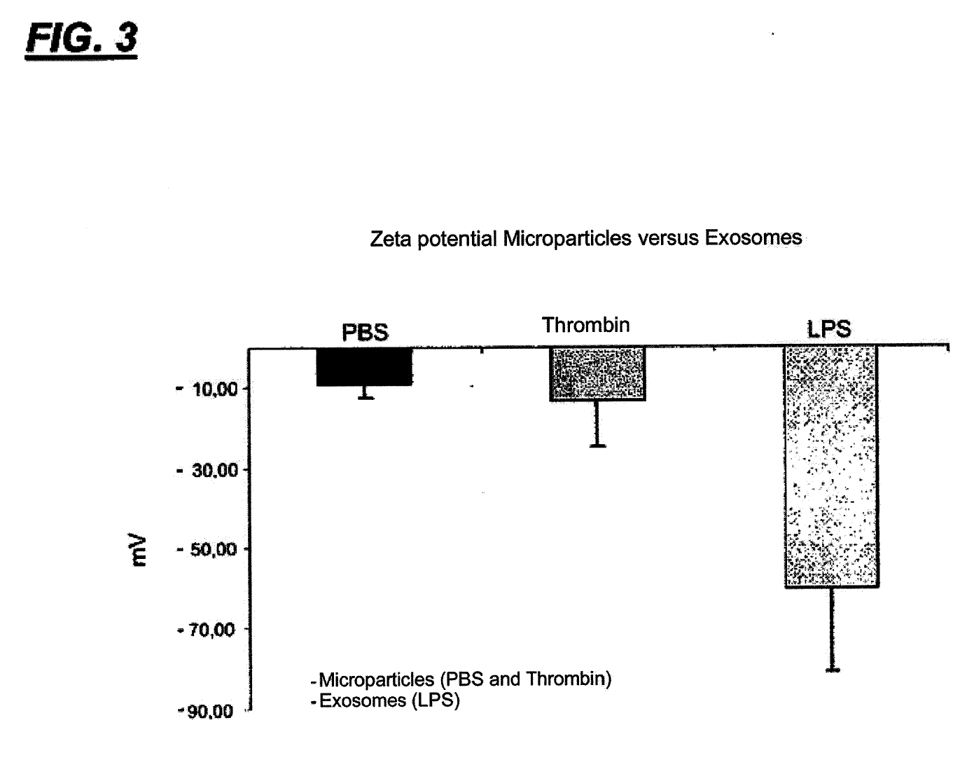

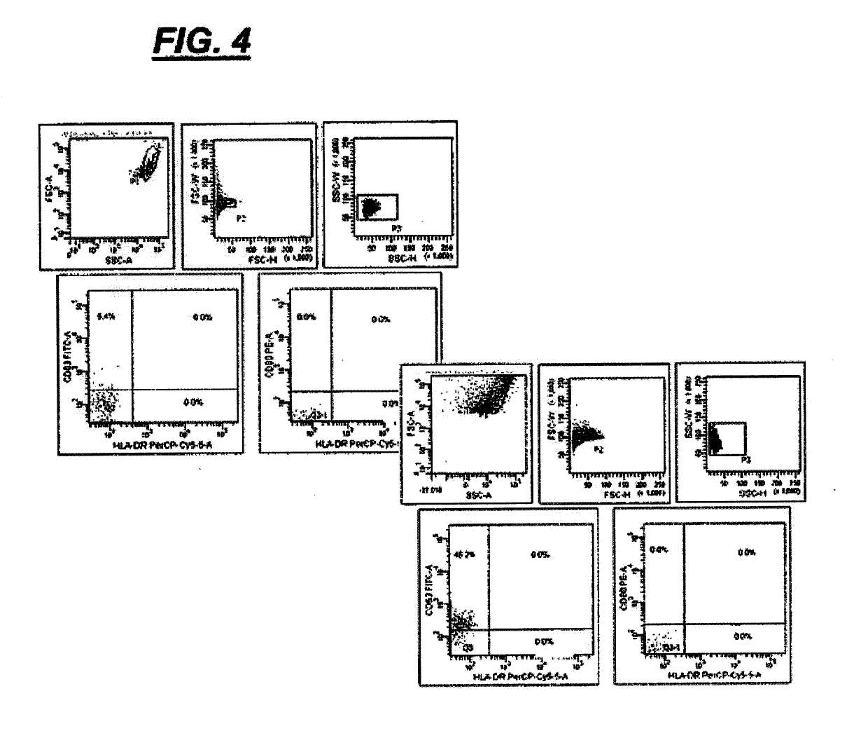

(a) Platelets were stimulated to generate typical exosomes and control particles
(b) Samples comprising exosomes (supernatant 2) and particles (supernatant 1) were submitted to zeta potential measure, revealing potential charges negative enough, but different among them (−61±21, 1) mV to the exosomes versus (−9, 2±3) mV to platelets degradation particles, average ±ep, n=4, p<0, 05);
(c) Solution of iron oxide superparamagnetic nanoparticles synthetized according to the methodology which basically consists in the rapid hydrolysis of Fe³⁺, by adding ammonium hydroxide in aqueous solution 0, 25 molar of FeCI₃.6H₂O; the precipitate dialysis allows a peptization leading to the formation of colloidal suspension with particles extremely small (˜200 Å);
(d) Nanometric particles (50-100 Å) based on iron oxide were prepared through alcoholic solutions hydrolysis by diethylammonium hydroxide in the presence of a surfactant as nonilfenol etoxilat;
(e) Samples were incubated with iron nanoparticles for 1 hour, proportion of 0, 1 ml concentration solution 200 μg of iron/mL for 2 ml of solution containing exosomes;
(f) After 1 hour, such material was exposed to a magnetic field in column LS-MidiMACS (Miltenyi) which allowed the split of the exosomes that were linked to the nanoparticles, by elution with PBS+mechanic force (piston of the column itself);
(g) Submitted to flow cytometry (CMF) is confirmed that exosomes were obtained (FIG. 3) through high expression of CD63 with some expression of CD9 and very low expression of Annexin V (FIG. 4)

The new method herein demonstrated, represents a significant advancement for the exosomes split due to the fact that its obtainment is significantly fast, through the manipulation of a unique solution, being the centrifugation and filtration not necessary and without pellet formation, that is polluted of dragged proteins during ultracentrifugation process and which ends altering the microparticles ultrastructure. On the other hand, ferrous nanoparticles are added in the middle. It is not clear yet if they are only linked to the external face of the exosomes or if they are incorporated by them. Such definition will allow subsequent development of the method for the split of ferrous material.

It is true that when this invention is put into practice alterations in reference to some construction and form details would be possible, without implying moving apart from the fundamental principles that are clearly mentioned in the claim table, being understood that the terminology used has the objective but not the limitation.

1. A method for isolation of exosomes from biological solutions using nanoparticles of iron oxide, featured by the fact that the method for exosomes isolation comprise platelets using superparamagnetic magnetite nanoparticles (Fe₃O₄), and be made through an attraction charge mechanism based on the exosomes pre-determined zeta potential where the iron oxide nanoparticles where previously synthetized with pre-determined positive charge, linked to the exosomes negatively charged, comprised in the biological sample, by means of electric attraction; the material exposure to a magnetic field allows the split of exosomes that were linked to the nanoparticles.

2. The method for isolation of exosomes from biological solutions using nanoparticles of iron oxide, in accordance with claim 1, featured by the method defined in the following stages:
(a) Platelets were stimulated to generate typical exosomes and control particles
(b) Samples comprising exosomes (supernatant 2) and particles (supernatant 1) were submitted to zeta potential measure, revealing potential charges negative enough, but different among them (−61±21, 1) mV to the exosomes versus (−9, 2±3) mV to platelets degradation particles, average ±ep, n=4, p<0, 05);
(c) Solution of iron oxide superparamagnetic nanoparticles synthetized according to the methodology which basically consists in the rapid hydrolysis of Fe³⁺, by adding ammonium hydroxide in aqueous solution 0, 25 molar of FeCI₃.6H₂O; the precipitate dialysis allows a peptization leading to the formation of colloidal suspension with particles extremely small (˜200 Å);
(d) Nanometric particles (50-100 Å) based on iron oxide were prepared through alcoholic solutions hydrolysis by diethylammonium hydroxide in the presence of a surfactant as nonilfenol etoxilat;
(e) Samples were incubated with iron nanoparticles for 1 hour, proportion of 0, 1 ml concentration solution 200 μg of iron/mL for 2 ml of solution containing exosomes;
(f) After 1 hour, such material was exposed to a magnetic field in column LS-MidiMACS (Miltenyi) which allowed the split of the exosomes that were linked to the nanoparticles, by elution with PBS+mechanic force (piston of the column itself);
(g) Submitted to flow cytometry (CMF) is confirmed that exosomes were obtained through high expression of CD63 with some expression of CD9 and very low expression of Annexin V

3. The method for isolation of exosomes from biological solutions using nanoparticles of iron oxide, in accordance with claims 1 featured by the fact that the method may obtain exosomes from mixed biological solutions.

4. The method for isolation of exosomes from biological solutions using nanoparticles of iron oxide, in accordance with claims 1 featured by the fact that the method may obtain undivided exosomes in their form.

5. The method for isolation of exosomes from biological solutions using nanoparticles of iron oxide, in accordance with claims 1 featured by the fact that the method may obtain undivided exosomes in their proteinic content.
